(19) United States
(12) Patent Application Publication
(10) Pub. No.: US 2024/0229111 A1
(43) Pub. Date: Jul. 11, 2024

(54) SYSTEMS AND METHODS FOR MODIFYING NUCLEIC ACID SEQUENCES TO ENHANCE COMPOUND PRODUCTION

(71) Applicant: Form Bio Inc., Austin, TX (US)

(72) Inventors: Andrew Busey, Austin, TX (US); Alexander Joseph Titus, Manchester, NH (US); Amicia Devin Elliott, Burien, WA (US); Joseph Charles Nipko, Boulder City, NV (US); Nicholas Ketz, Madison, WI (US); Alexandra Maslova, Vancouver (CA)

(21) Appl. No.: 18/150,404

(22) Filed: Jan. 5, 2023

(51) Int. Cl. C12Q 1/6827

(52) U.S. Cl. CPC C12Q 1/6827; C12Q 2600/154

(57) ABSTRACT

System and method for modifying nucleic acid sequences to enhance compound production are disclosed. Exemplary implementations may: obtain genomic information including a representation of a nucleic acid sequence that includes an order of nucleotides; identify individual promoter regions in the order of nucleotides; detect one or more CpG islands; determine one or more probabilities of methylation for individual CpG islands; select one or more of the individual CpG islands for modification of one or more CpG pairs included in the one or more of the individual CpG islands; identify one or more alternative orders of nucleotides for the one or more CpG pairs of the selected CpG islands; analyze the one or more alternative orders of nucleotides; present the one or more alternative orders of nucleotides; and/or perform other steps.

### FIELD OF THE DISCLOSURE

The present disclosure relates to systems and methods for modifying nucleic acid sequences, e.g., of a genome, to enhance compound production, including but not limited to protein.

### BACKGROUND

Methods for determining and analyzing genomic information are known (e.g., DNA sequencing, etc.). Genomic information may be tested using various programs and/or methods for accuracy and/or quality prior to compound production.

### SUMMARY

Compound production (including but not limited to protein synthesis) may pose many challenges. Genomic information including representations of nucleic acid sequences may provide the blueprint to produce amino acids and/or other structures for compound production. Phenomena present in or occurring to the genomic information may prevent effective compound production, produce defective compounds and/or structures, and/or decrease the speed of compound production. Therefore, reduction of these phenomena by modifying (i.e., editing) the genomic information may be appropriate prior to compound production. Reduction of these phenomena may enhance compound production and/or result in a higher quality product, however it may not be possible to eliminate all instances or occurrences of these phenomena. Attempting to eliminate one instance or occurrence of a phenomena may result in creation of one or more others and/or modify the amino acids produced by the genomic information.

One or more aspects of the present disclosure include a system for modifying nucleic acid sequences to enhance compound production. The system may include electronic storage, one or more hardware processors configured by machine-readable instructions, and/or other components. Executing the machine-readable instructions may cause the one or more hardware processors to facilitate modifying nucleic acid sequences to enhance compound production. The system may be configured to obtain genomic information including a representation of a nucleic acid sequence that includes an order of nucleotides (i.e., a particular sequence of nucleotides). The system may be configured to identify individual promoter regions in the order of nucleotides. The system may be configured to detect one or more cytosine-phosphate-guanine (CpG) islands. The system may be configured to determine one or more probabilities of methylation for individual CpG islands (i.e., where a methyl group couples with a cytosine). The system may be configured to select one or more of the individual CpG islands for modification of one or more CpG pairs included in the one or more of the individual CpG islands. The system may be configured to identify one or more alternative orders of nucleotides for the selected CpG islands (in other words, one or more alternative sequences of nucleotides for the one or more CpG pairs of the selected CpG islands). The system may be configured to analyze the one or more alternative orders of nucleotides. The system may be configured to present the one or more alternative orders of nucleotides. The system may be configured to perform other steps.

One or more aspects of the present disclosure include a method of modifying nucleic acid sequences to enhance compound production. The method may include obtaining genomic information including a representation of a nucleic acid sequence that includes an order of nucleotides. The method may include identifying individual promoter regions in the order of nucleotides. The method may include detecting one or more CpG islands. The method may include determining one or more probabilities of methylation for individual CpG islands. The method may include selecting one or more of the individual CpG islands for modification of one or more CpG pairs included in the one or more of the individual CpG islands. The method may include identifying one or more alternative orders of nucleotides for the one or more CpG pairs of the selected CpG islands. The method may include analyzing the one or more alternative orders of nucleotides. The method may include presenting the one or more alternative orders of nucleotides. The method may include other steps.

As used herein, any association (or relation, or reflection, or indication, or correspondency, or correlation) involving servers, processors, client computing platforms, users, representations, sequences, promoter regions, CpG islands, determinations, detections, selections, rankings, probabilities, modifications, presentations, interfaces, and/or another entity or object that interacts with any part of the system and/or plays a part in the operation of the system, may be a one-to-one association, a one-to-many association, a many-to-one association, and/or a many-to-many association or “N”-to-“M” association (note that “N” and “M” maybe different numbers greater than 1).

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, local and/or remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system for modifying nucleic acid sequences to enhance compound production, in accordance with one or more implementations.

FIG. 2 illustrates a method for modifying nucleic acid sequences to enhance compound production, in accordance with one or more implementations.

FIG. 3 illustrates an exemplary user interface for use with a system configured for modifying nucleic acid sequences to enhance compound production, in accordance with one or more implementations.

FIG. 4 illustrates exemplary orders of nucleotides as may be used by a system configured for modifying nucleic acid sequences to enhance compound production, in accordance with one or more implementations.

### DETAILED DESCRIPTION

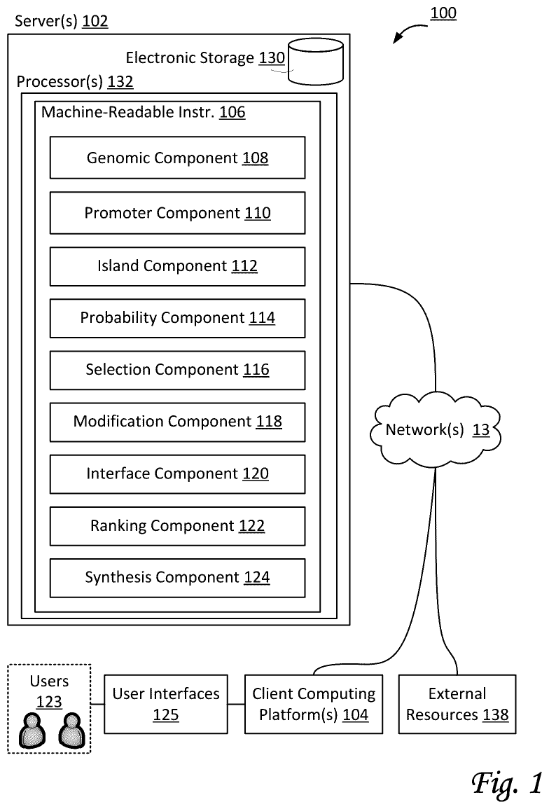

FIG. 1 illustrates a system 100 configured for modifying nucleic acid sequences to enhance compound production, in accordance with one or more implementations. In some implementations, system 100 may include one or more servers 102, one or more client computing platforms 104, one or more external resources 138, and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users 123 may access system 100 via client computing platform(s) 104, e.g., using one or more user interfaces 125 associated with (or included in) one or more client computing platforms 104.

Server(s) 102 may be configured by machine-readable instructions 106. Machine-readable instructions 106 may include one or more instruction components. The instruction components may include computer program components. The instruction components may include one or more of a genomic component 108, a promoter component 110, an island component 112, a probability component 114, a selection component 116, a modification component 118, an interface component 120, a ranking component 122, a synthesis component 124, and/or other instruction components.

Genomic component 108 may be configured to obtain genomic information and/or other information. The genomic information may include a representation of one or more nucleic acid sequences (e.g., of a genome), and/or other structures. The representation may include an order of nucleotides that defines a nucleic acid sequence. The genomic information may include a FASTA file, FASTQ file, BAM file, SAM file, BAS file, and/or other file types for representing the one or more nucleic acid sequences and/or other structures. The nucleic acid sequence may be a deoxyribonucleic acid (DNA) sequence, a ribonucleic acid (RNA) sequence, an mRNA sequence, tRNA sequence, and/or other types of nucleic acid sequences. In some implementations, genomic component 108 may convert the genomic information from a first file type to a second file type to facilitate identification of instances of phenomena, modification of the representations of the nucleic acid sequence, and/or other functions. Genomic component 108 may convert the genomic information from a file type that is incompatible with other components of system 100 to a file type that is compatible.

The order of nucleotides may include representations of adenine (A), thymine (T), guanine (G), cytosine (C), uracil (U), and/or other nucleotides. The nucleic acid sequence and/or the order of nucleotides that defines the nucleic acid sequence may result in synthesis of one or more compounds (e.g., amino acids). Compound production may include protein and/or different functional gene products, such as, e.g., functional non-doing RNA. In some implementations, the representation of the nucleic acid sequence may include one or more pairs of orders of nucleotides. For example, a pair of orders of nucleotides includes a first order of nucleotides and a second order of nucleotides. One or more base pairs may include a nucleotide at a first position on the first order of nucleotides and a nucleotide at the same (corresponding) first position on the second order of nucleotides. Base pairs may be defined by the type of nucleotides included in the base pair (e.g., AU, AT, GC), a location or position on the pair of orders of nucleotides (e.g., an index), and/or other information. Base pairs may be formed of a pair of nucleotides within the same order of nucleotides. For example, a base pair may include a first nucleotide and a second nucleotide on the first order of nucleotides. The base pair may be formed during folding of the nucleic acid sequence and/or other processes of compound synthesis. In some implementations, folding may occur during formation of compact structures (e.g., formation of chromatin).

In some implementations, the genomic information may further include quality information associated with the one or more nucleic acid sequences. In some implementations, the quality information may include one or more of a quality score, integrity number and/or other information. The quality score may indicate a level of confidence in the accuracy in the representation of the nucleic acid sequence and/or other information. The quality information may be obtained from the user and/or imported (e.g., via client computing platform(s) 104, external resources 138). The genomic information may include indexing information associated with the one or more orders of nucleotides. The indexing information may indicate positions (i.e., indexes) on the order of nucleotides, the type of nucleotide(s) (e.g., A, U, T, G, C) found at the position, and/or other information. In some implementations, the indexing information may be obtained with the genomic information. In some implementations, the indexing information may be generated by genomic component 108 based on the obtained genomic information.

The order of nucleotides may include instances of phenomena and/or other components and/or structures. The types of phenomena may include CpG islands, (high) truncation propensity, (high) methylation propensity, transcription factor binding sites, and/or other types of phenomena. An instance of a CpG island may be characterized by a concentration of representations of cytosine and/or guanine within a given segment of the order of nucleotides exceeding a threshold. The given segment may have a length of 300 nucleotides, 1000 nucleotides, 3000 nucleotides, and/or other lengths of nucleotides. The given segment may have a length of 300 base pairs, 1000 base pairs, 3000 base pairs, and/or other lengths of base pairs. In some implementations, the threshold may be a number of C or G nucleotides, a number of GC base pairs (or CpG pairs), a percentage of C or G nucleotides, a percentage of GC base pairs (or CpG pairs), and/or other values. The percentage of C or G nucleotides and/or GC base pairs may be 40%, 50%, 55%, 60%, 65%, and/or other percentages. By way of non-limiting example, a segment of base pairs may be characterized as a CpG island responsive to 55% or more of the base pairs within the segment being GC base pairs.

An instance of a truncation propensity may be characterized by an ordered sequence of three nucleotides predicted to shorten the nucleic acid sequences (i.e., effectuates termination of compound synthesis). In some implementations, a list of known truncation variants (i.e., stop codons) may be obtained from one or more of electronic storage 130, external resources 138, and/or other components of system 100. In some implementations, a list of truncation variants may be obtained from the user, e.g., through user interfaces 125. Individual nucleotide triplets in the nucleic acid sequence may be compared to the individual truncation variants of the one or more lists to identify instances of truncation propensity.

An instance of high methylation propensity may be characterized by an ordered sequence of nucleotides predicted (i.e., corresponding to a prediction that meets or exceeds a threshold) to attach to a methyl group. An instance of a transcription factor binding site may be characterized by an order sequence of nucleotides that impacts and/or regulates gene expression. In some implementations, a list of sequences defining known transcription factor binding sites may be obtained from the user and/or external resources 138. Individual segments in the nucleic acid sequence may be compared to the list of sequences of known transcription factor binding sites to identify instances of transcription factor binding sites.

Genomic component 108 may be configured to obtain user-defined priority information and/or other information. In some implementations, the user-defined priority information may be obtained via a user interface of client computing platform(s) 104 and/or interface component 120. The user-defined priority information may indicate an order (i.e., ranking) of types of phenomena. The order and/or ranking of the types of phenomena may indicate relative priority of eliminating individual ones of the types of phenomena present in the order of nucleotides that define the nucleic acid sequence. For example, the priority information may indicate a first ranking for a first type of phenomena and a second ranking for a second type of phenomena. The rankings may indicate a higher prioritization for eliminating instances of the first type of phenomena compared to the second type of phenomena. Based on the rankings, a modification to the order of nucleotides that eliminates the first type of phenomena may be preferred over a modification that eliminates the second type of phenomena.

Promoter component 110 may be configured to identify and/or obtain individual promoter regions in orders of nucleotides. In particular, promoter component 110 may identify and/or obtain one or more individual promoter regions in the particular order of nucleotides that is included in a particular representation as obtained by genomic component 108. In some implementations, an individual promoter region may serve to initiate transcription, e.g., of DNA into RNA.

Island component 112 may be configured to detect, identify, and/or obtain CpG islands, e.g., in orders of nucleotides. For example, island component 112 may detect a set of CpG islands in a particular promoter region (e.g., based on operations of promoter component 110). In some implementations, instances of CpG islands may be characterized by a concentration of cytosine and/or guanine exceeding a threshold (e.g., a threshold concentration of 50%, or 55%, or 60%, or 65%, or 70%, or 75%, or 80%, or 85%, or 90%, or 95%, or another percentage). In some implementations, instances of CpG islands may be characterized by start positions and end positions in a particular nucleic acid sequence.

Probability component 114 may be configured to determine probabilities of methylation for individual CpG islands, for individual CpG pairs in a CpG island, for promoter regions, for order of nucleotides, and/or for (parts of) nucleic acid sequences. In some implementations, probability component 114 may be configured to determine one or more probabilities of methylation for an individual CpG island (such as, e.g., a CpG island as detected by island component 112). For example, probability component 114 may determine a set of probabilities pertaining to methylation for a particular CpG island. Accordingly, a probability or set of probabilities may be island-specific. In some cases, a particular probability may be specific to a CpG pair. Determinations by probability component 114 may be based on context information of individual CpG islands within a particular the nucleic acid sequence. For example, the context information for an individual CpG island includes index information that indicates a start position and an end position within a nucleic acid sequence. In some implementations, context information may include information regarding phenomena within an individual CpG island, or near an individual CpG island. Alternatively, and/or simultaneously, context information may include any characteristics of an individual CpG island that negatively affect compound production, including but not limited to characteristics that cause a CpG island to be methylation-prone. Probability component 114 may be configured to aggregate multiple individual probabilities into a single probability, e.g., through statistical operators or operations. By way of non-limiting example, two different probabilities could be averaged into a single probability.

In some implementations, island component 112, probability component 114, and/or another component of system 100 may be configured to further select, filter, and/or otherwise restrict a set of CpG islands (e.g., in a particular promoter region) into a smaller subset of CpG islands. For example, by restricting operations to a particular subset of CpG islands, the determination of one or more probabilities by probability component 114 may be restricted to the particular subset. In some implementations, this restriction may be based on downstream effects of methylation, including but not limited to whether the genes and/or compounds are significantly altered by the methylation. In some implementations, this restriction may be based on heuristics regarding production efficiency. In some implementations, this restriction may be based on input from a user, such as, e.g., user-provided filters and/or user-provided criteria. In some implementations, this restriction may be performed automatically.

In some implementations, determinations by probability component 114 may be based on real-world measurements, including but not limited to measurements resulting from bench-scale tests of nucleic acid sequences in compound production. Characterizing compounds may include inspecting the individual alternative nucleic acid sequences to determine the compounds that may be produced, and/or identify occurrences (e.g., reactions, outputs, byproducts, etc.) which may result from production of compounds. The results of bench-scale tests may include one or more of a compound (e.g., protein) yield rate, a compound concentration, a number of secondary structures formed, a translation rate, a gene suppression rate, an energy consumption rate, a concentration of vector genome and viral particles (i.e., capsid titer), a protein expression rate, enzyme activity, information characterizing protein structures, and/or other metrics and/or information obtained from bench-scale tests. In some implementations, (user-provided) criteria may be defined by these values of results of bench-scale tests for nucleic acid sequences. By way of non-limiting example, one of the (user-provided) criteria may be defined by a compound yield rate of 2 mg/ml (i.e., two milligrams of compound per one milliliter of solution), and/other values of results of bench-scale tests.

In some implementations, bench-scale tests of individual alternative nucleic acid sequences may be performed in one or more bioreactors and/or other devices and/or structures that may support a biologically active environment. Devices and/or structures that may support a biologically active environment may be capable of supporting chemical and biological processes. The one or more bioreactors may be configured to replicate conditions of large-scale production of compounds. In some implementations, conditions within the bioreactor may be modified. The conditions may include one or more of temperature, pH level, hydrodynamic stresses, chemical species, agitation and aeration, biochemical kinetics, atmosphere conditions (e.g., composition of atmosphere including carbon dioxide, oxygen, etc.), humidity, cell culture types (e.g., HEK293-T, CHO, etc.), transfection agents and transduction agents (e.g., plasmid for viral packaging, etc.) and/or other conditions. The results of bench-scale tests may be predictive of the performance of particular alternative nucleic acid sequences in large-scale production of compounds.

In some implementations, determinations by probability component 114 may be based on predictions generated by a model. In some implementations, the model may be a (trained) machine learning model. In some implementations, the model may use heuristics to generate predictions (e.g., an expert system may be used to generate predictions). For example, a particular model may be stored in electronic storage 130.

Selection component 116 may be configured to select CpG islands for modification of one or more CpG pairs included in the CpG islands. In some implementations, selection component 116 may be configured to select one or more CpG pairs from a particular CpG island for modification of the one or more CpG pairs included in the particular CpG island. In some implementations, selection by selection component 116 may be based on one or more probabilities such as, e.g., probabilities determined by probability component 114. Alternatively, and/or simultaneously, selection by selection component 116 may be based on one or more user-provided criteria. In some implementations, the selection may use a threshold of determined methylation probability, such as, say, CpG islands with 80% or more methylation (or rather, 80% probability of significant methylation that negatively affects compound production). This specific percentage is merely exemplary.

Modification component 118 may be configured to generate and/or identify alternative orders of nucleotides for CpG pairs and/or CpG islands. In particular, modification component 118 may identify one or more alternative orders of nucleotides for those CpG pairs and/or CpG islands that have been selected using selection component 116 for modification. In some implementations, generating a particular alternative order of nucleotides (for a particular order of nucleotides) may include modifying and/or replacing one or more nucleotides in the particular order of nucleotides (particularly, for one or more CpG pairs). For example, modifications may be based on identified instances of phenomena, phenomena information, indexing information, obtained genomic information, and/or other (context) information. In some implementations, responsive to removal of one or more nucleotides in an order of nucleotides, genomic component 108 may update the indexing information, the phenomena information, and/or other information in accordance with the modification.

In some implementations, probability component 114 may be configured to analyze alternative orders of nucleotides. In some implementations, analyzing alternative orders of nucleotides may include determining one or more methylation probabilities for individual ones of the alternative orders of nucleotides. For example, analysis might show that a particular alternative order of nucleotides has a lower methylation probability than the corresponding (original) order of nucleotides that was selected for modification. For example, analysis might show that a particular alternative order of nucleotides has a lower probability of significant methylation that negatively affects compound production than the corresponding (original) order of nucleotides that was selected for modification.

Interface component 120 may be configured to present alternative orders of nucleotides and/or other information, e.g., to users. In some implementations, interface component 120 may present alternative orders of nucleotides and/or other information on user interfaces 125, for example, using one or more (ordered and/or otherwise organized) lists and/or other formats. In some implementations, interface component 120 may generate and present a summary of information to the user using a graph, chart, plot, and/or other types of visual summaries.

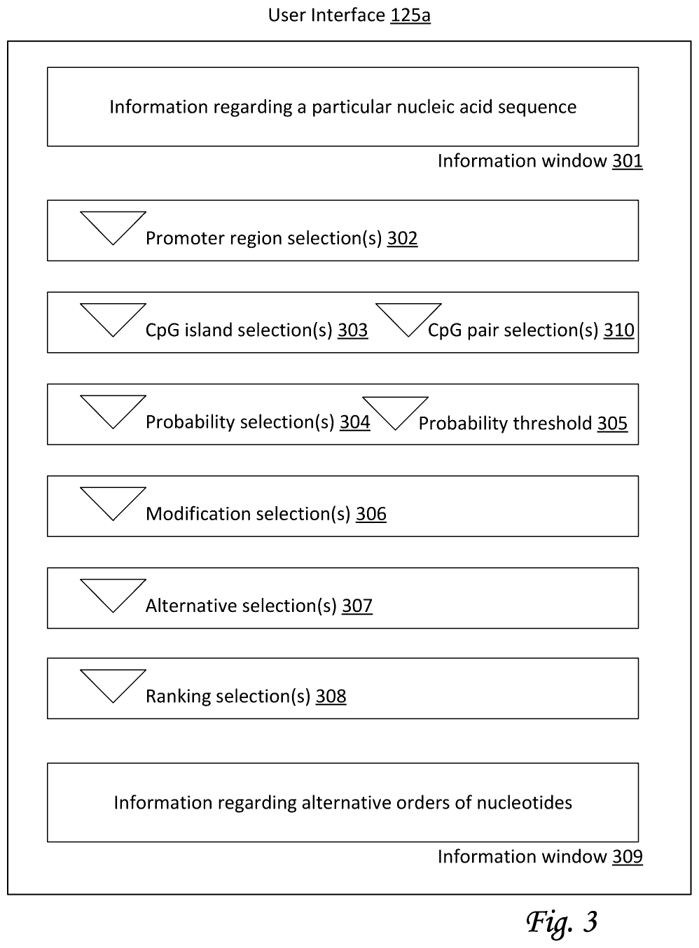

By way of non-limiting example, FIG. 3 illustrates an exemplary user interface 125a for use with system 100. User interface 125a may be presented to a user, e.g., via a client computing platform associated with the user. User interface 125a may include sections or fields for presenting information, interaction with a user, and/or other graphical user interface elements. As depicted, user interface 125a may include an information window 301 to present information regarding a particular nucleic acid sequence. User interface 125a may include a graphical user interface element for promoter region selection(s) 302, through which a user may enter and/or select information used to select, filter, and/or otherwise limit the promoter regions to be used by system 100 for the modification of nucleic acid sequences to enhance compound production. User interface 125a may include a graphical user interface element for CpG island selection(s) 303, through which a user may enter and/or select information used to select, filter, and/or otherwise limit the CpG islands to be used by system 100 for the modification of nucleic acid sequences to enhance compound production. User interface 125a may include a graphical user interface element for CpG pair selection(s) 310, through which a user may enter and/or select information used to select, filter, and/or otherwise limit the CpG pairs to be used by system 100 for the modification of nucleic acid sequences to enhance compound production. User interface 125a may include a graphical user interface element for probability selection(s) 304, through which a user may enter and/or select information used to select, filter, and/or otherwise limit the type of probability or probabilities to be determined by system 100 for the modification of nucleic acid sequences to enhance compound production. For example, one or more of probability selection(s) 304 may be used to select which types of context information is taken into account for the determinations of probabilities. User interface 125a may include a graphical user interface element for probability threshold 305, through which a user may enter and/or select information used to select the probability threshold to be used by system 100 for the modification of nucleic acid sequences to enhance compound production. User interface 125a may include a graphical user interface element for modification selection(s) 306, through which a user may enter and/or select information used to identify, select, filter, and/or otherwise limit the CpG islands to be modified by system 100. User interface 125a may include a graphical user interface element for alternative selection(s) 307, through which a user may enter and/or select information used to select, filter, and/or otherwise limit the alternative orders of nucleotides to be used by system 100 for the modification of nucleic acid sequences to enhance compound production. For example, one or more of alternative selection(s) 307 may be used to select which sources of alternative orders of nucleotides are to be used by system 100, including but not limited to heuristic models, machine learning models, user-provided orders of nucleotides, and/or other sources. User interface 125a may include a graphical user interface element for ranking selection(s) 308, through which a user may enter and/or select information used to order, rank, and/or otherwise organize alternative orders of nucleotides for presentation to the user. As depicted, user interface 125a may include an information window 309 to present information regarding alternative orders of nucleotides, and, in particular, the alternative orders of nucleotides after one or more of promoter region selection(s) 302, CpG island selection(s) 303, CpG pair selection(s) 310, probability selection(s) 304, probability threshold 305, modification selection(s) 306, alternative selection(s) 307, and/or ranking selection(s) 308 have been entered by the user and applied or used by system 100.

Referring to FIG. 1, ranking component 122 may be configured to rank (a set of) alternative orders of nucleotides, e.g., based on an analysis from probability component 114. In some implementations, a ranking may be based on one or more probabilities as determined by probability component 114. Alternatively, and/or simultaneously, a ranking may be based on user-provided criteria. In some implementations, a particular presentation of one or more alternative orders of nucleotides (e.g., by interface component 120) may be performed according to a particular ranking as determined by ranking component 122.

Synthesis component 124 may be configured to facilitate synthesis of some or all of a particular nucleic acid sequence. For example, the particular nucleic acid sequence may include at least one of the alternative orders of nucleotides as identified. In some implementations, the particular nucleic acid sequence may include at least one of the alternative orders of nucleotides as identified for at least one of the individual CpG pairs and/or CpG islands as selected for modification. In some implementations, synthesis component 124 may be configured to measure production efficiency, e.g., of a particular type of synthesis.

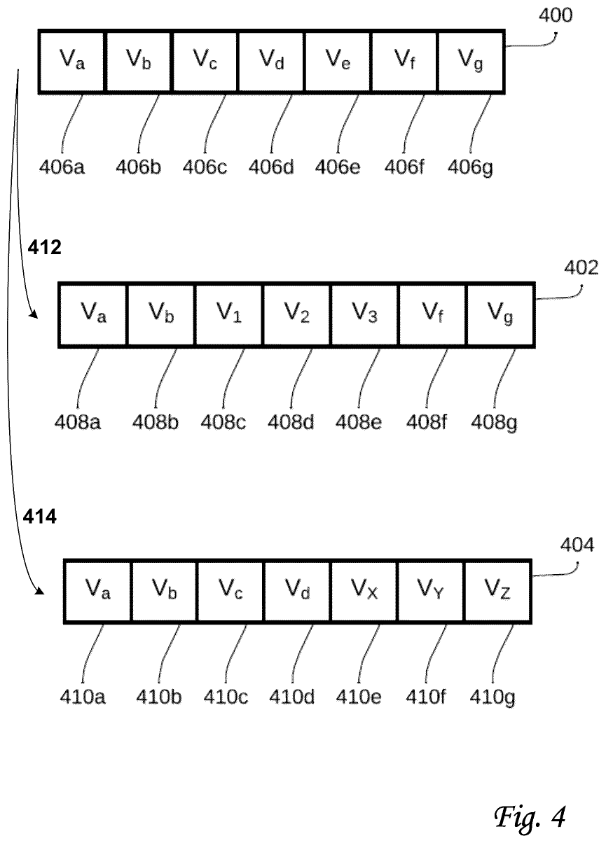

FIG. 4 illustrates exemplary orders of nucleotides as may be used by a system configured for modifying nucleic acid sequences to enhance compound production. FIG. 4 shows a first order of nucleotides 400 having representations of nucleotides 406a-g. The representations of nucleotides 406a-g having values Vₐ-V_{g}, respectively. In some implementations, first order of nucleotides may include a first instance of a phenomenon that negatively affects (or might negatively affect) compound production and/or other instances of phenomena. One or more modifications of first order of nucleotides 400 may be performed to eliminate the first instance of a particular phenomenon. For example, a first modification 412 (represented by an arrow) may generate a first alternative order of nucleotides 402 and/or other alternative orders of nucleotides. First alternative order of nucleotides 402 may be generated by replacing values V_{c}-V_{d}-Vₑ (representations of nucleotides 406c-406d-406e) of first order of nucleotides 400 with values V₁-V₂-V₃ (representations of nucleotides 408c-408d-408e). A second modification 414 (represented by an arrow) may generate a second alternative order of nucleotides 404 and/or other alternative orders of nucleotides. Second alternative order of nucleotides 404 may be generated by replacing values Vₑ-V_{f}-V_{g} (representations of nucleotides 406e-406f-406g) of first order of nucleotides 400 with values V_{X}-V_{Y}-V_{Z} (representations of nucleotides 410e-410f-410g). First alternative order of nucleotides 402, second alternative order of nucleotides 404, and/or other alternative orders of nucleotides may not include one or more particular instances of phenomena that are present in first order of nucleotides 400.

Referring to FIG. 1, in some implementations, server(s) 102, client computing platform(s) 104, and/or external resources 138 may be operatively linked via one or more electronic communication links. For example, such electronic communication links may be established, at least in part, via one or more (electronic communication) networks 13, which may include the Internet and/or other networks. It will be appreciated that this is not intended to be limiting, and that the scope of this disclosure includes implementations in which server(s) 102, client computing platform(s) 104, and/or external resources 138 may be operatively linked via some other communication media.

A given client computing platform 104 may include one or more processors configured to execute computer program components. The computer program components may be configured to enable an expert or user associated with the given client computing platform 104 to interface with system 100 and/or external resources 138, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms. Client computing platforms 104 may be associated with (or may include) user interfaces 125.

External resources 138 may include sources of information outside of system 100, external entities participating with system 100, and/or other resources. In some implementations, some or all of the functionality attributed herein to external resources 138 may be provided by resources included in system 100. In some implementations, external resources 138 may include one or more bioreactors. In some implementations, external resources 138 may include one or more (trained machine learning) models.

Server(s) 102 may include electronic storage 130, one or more processors 132, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 130 may comprise non-transitory storage media that electronically stores information. The electronic storage media of electronic storage 130 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may include one or more virtual storage resources (e.g., cloud storage, a virtual private network, and/or other virtual storage resources). Electronic storage 130 may store software algorithms, information determined by processor(s) 132, information received from server(s) 102, information received from client computing platform(s) 104, (machine learning) models, and/or other information that enables server(s) 102 and/or system 100 to function as described herein.

Processor(s) 132 may be configured to provide information processing capabilities in server(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 1 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute components 108, 110, 112, 114, 116, 118, 120, 122, and/or 124, and/or other components. Processor(s) 132 may be configured to execute components 108, 110, 112, 114, 116, 118, 120, 122, and/or 124, and/or other components by software; hardware; firmware; some combination of software, hardware, and/or firmware; and/or other mechanisms for configuring processing capabilities on processor(s) 132. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112 and/or 114 are illustrated in FIG. 1 as being implemented within a single processing unit, in implementations in which processor(s) 132 includes multiple processing units, one or more of components 108, 110, 112 and/or 114 may be implemented remotely from the other components. The description of the functionality provided by the different components 108, 110, 112 and/or 114 described below is for illustrative purposes, and is not intended to be limiting, as any of components 108, 110, 112 and/or 114 may provide more or less functionality than is described. For example, one or more of components 108, 110, 112 and/or 114 may be eliminated, and some or all of its functionality may be provided by other ones of components 108, 110, 112, 114, 116, 118, 120, 122, and/or 124. As another example, processor(s) 132 may be configured to execute one or more additional components that may perform some or all of the functionality attributed below to one of components 108, 110, 112, 114, 116, 118, 120, 122, and/or 124.

Referring to FIG. 1, user interfaces 125 may be configured to facilitate interaction between users 123 and system 100 and/or between users 123 and client computing platforms 104. For example, user interfaces 125 may provide an interface through which users 123 may provide information to and/or receive information from system 100. In some implementations, user interface 125 may include one or more of a display screen, touchscreen, monitor, a keyboard, buttons, switches, knobs, levers, mouse, microphones, sensors to capture voice commands, sensors to capture body movement, sensors to capture hand and/or finger gestures, and/or other user interface devices configured to receive and/or convey user input. In some implementations, one or more user interfaces 125 may be included in one or more client computing platforms 104. In some implementations, one or more user interfaces 125 may be included in system 100.

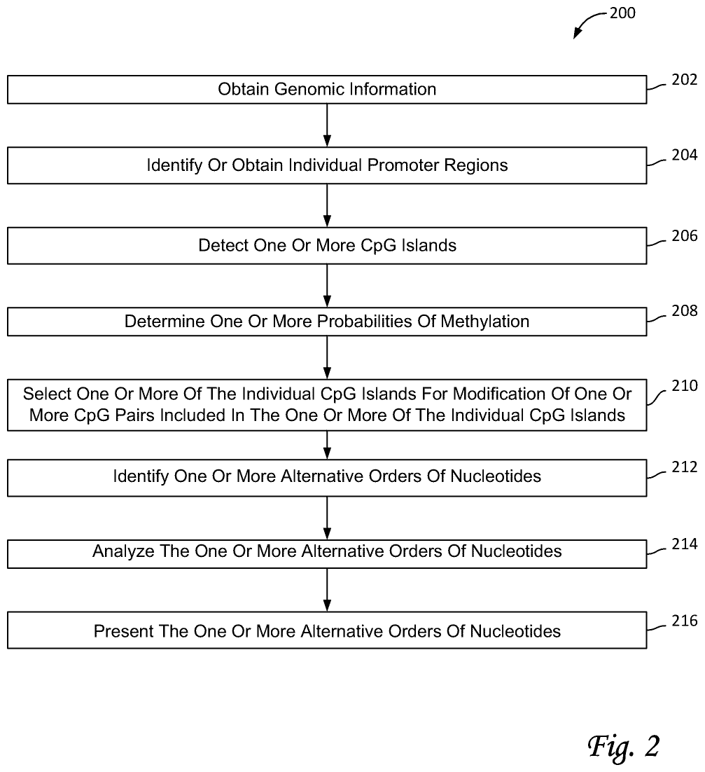

FIG. 2 illustrates a method 200 for modifying nucleic acid sequences to enhance compound production, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, genomic information is obtained. The genomic information includes a representation of a nucleic acid sequence. The representation includes an order of nucleotides that defines the nucleic acid sequence. The nucleic acid sequence results in synthesis of one or more compounds during the compound production. In some embodiments, operation 202 is performed by a genomic component the same as or similar to genomic component 108 (shown in FIG. 1 and described herein).

At an operation 204, individual promoter regions are identified or obtained in the order of nucleotides. Individual ones of the promoter regions serve to initiate transcription. In some embodiments, operation 204 is performed by a promoter component the same as or similar to promoter component 110 (shown in FIG. 1 and described herein).

At an operation 206, for individual ones of the promoter regions, one or more CpG islands are detected. Instances of CpG islands are characterized by a concentration of cytosine and/or guanine exceeding a threshold. The instances of the CpG islands are characterized by start positions and end positions in the nucleic acid sequence. In some embodiments, operation 206 is performed by an island component the same as or similar to island component 112 (shown in FIG. 1 and described herein).

At an operation 208, for individual CpG islands as detected, one or more probabilities of methylation are determined. The determination is based on context information of the individual CpG islands within the nucleic acid sequence. In some embodiments, operation 208 is performed by a probability component the same as or similar to probability component 114 (shown in FIG. 1 and described herein).

At an operation 210, one or more of the individual CpG islands are selected for modification of one or more CpG pairs included in the one or more of the individual CpG islands. In some embodiments, operation 210 is performed by a selection component the same as or similar to selection component 116 (shown in FIG. 1 and described herein).

At an operation 212, for individual CpG islands as selected for modification of the one or more CpG pairs, one or more alternative orders of nucleotides for the one or more CpG pairs are identified. The one or more alternative orders of nucleotides result in the synthesis of the same one or more compounds as the order of nucleotides. In some embodiments, operation 212 is performed by a modification component the same as or similar to modification component 118 (shown in FIG. 1 and described herein).

At an operation 214, the one or more alternative orders of nucleotides are analyzed. Analyzing the one or more alternative orders of nucleotides includes determining one or more methylation probabilities for individual ones of the one or more alternative orders of nucleotides. In some embodiments, operation 214 is performed by a probability component the same as or similar to probability component 114 (shown in FIG. 1 and described herein).

At an operation 216, the one or more alternative orders of nucleotides are presented to a user. In some embodiments, operation 216 is performed by an interface component the same as or similar to interface component 120 (shown in FIG. 1 and described herein).

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed:

1. A system configured to modify nucleic acid sequences to enhance compound production, the system comprising:
one or more processors configured by machine readable instructions configured to:
obtain genomic information, wherein the genomic information includes a representation of a nucleic acid sequence, wherein the representation includes an order of nucleotides that defines the nucleic acid sequence, wherein the nucleic acid sequence results in synthesis of one or more compounds during the compound production;
identify or obtain individual promoter regions in the order of nucleotides, wherein individual ones of the promoter regions serve to initiate transcription;
for individual ones of the promoter regions, detect one or more CpG islands, wherein instances of CpG islands are characterized by a concentration of cytosine and/or guanine exceeding a threshold, and wherein the instances of the CpG islands are characterized by start positions and end positions in the nucleic acid sequence;
for individual CpG islands as detected, determine one or more probabilities of methylation, wherein the determination is based on context information of the individual CpG islands within the nucleic acid sequence;
select one or more of the individual CpG islands for modification of one or more CpG pairs included in the one or more of the individual CpG islands;
for individual CpG islands as selected for modification of the one or more CpG pairs, identify one or more alternative orders of nucleotides for the one or more CpG pairs, wherein the one or more alternative orders of nucleotides result in the synthesis of the same one or more compounds as the order of nucleotides;
analyze the one or more alternative orders of nucleotides, wherein analyzing the one or more alternative orders of nucleotides includes determining one or more methylation probabilities for individual ones of the one or more alternative orders of nucleotides; and
present the one or more alternative orders of nucleotides.

2. The system of claim 1, wherein selection of the individual CpG islands is based on the one or more probabilities as determined.

3. The system of claim 1, wherein selection of the individual CpG islands is based on user-provided criteria.

4. The system of claim 1, wherein the one or more processors are further configured to:
for the individual CpG islands as detected, select a subset of CpG islands based on significance of downstream effects of methylation,
wherein the determination of the one or more probabilities of methylation is restricted to the subset of CpG islands.

5. The system of claim 1, wherein the determination of the one or more probabilities of methylation is further based on real-world measurements.

6. The system of claim 1, wherein the determination of the one or more probabilities of methylation is further based on predictions generated by a model.

7. The system of claim 1, wherein the one or more processors are further configured to:
rank the one or more alternative orders of nucleotides based on the analysis, and wherein the presentation of the one or more alternative orders of nucleotides is performed according to the ranking.

8. The system of claim 1, wherein identifying the one or more alternative orders of nucleotides for the one or more CpG pairs includes modifying and/or replacing one or more nucleotides in the one or more CpG pairs.

9. The system of claim 1, wherein the context information for an individual CpG island includes index information that indicates a start position and an end position within the nucleic acid sequence.

10. The system of claim 1, wherein the one or more compounds include one or more proteins and/or functional non-coding ribonucleic acid (RNA).

11. The system of claim 1, wherein the one or more processors are further configured to:
facilitate synthesis of some or all of the nucleic acid sequence, including at least one of the alternative orders of nucleotides as identified.

12. A method of modifying nucleic acid sequences to enhance compound production, the method comprising:
obtaining genomic information, wherein the genomic information includes a representation of a nucleic acid sequence, wherein the representation includes an order of nucleotides that defines the nucleic acid sequence, wherein the nucleic acid sequence results in synthesis of one or more compounds during the compound production;
identifying or obtaining individual promoter regions in the order of nucleotides, wherein individual ones of the promoter regions serve to initiate transcription;
for individual ones of the promoter regions, detecting one or more CpG islands, wherein instances of CpG islands are characterized by a concentration of cytosine and/or guanine exceeding a threshold, and wherein the instances of the CpG islands are characterized by start positions and end positions in the nucleic acid sequence;
for individual CpG islands as detected, determining one or more probabilities of methylation, wherein the determination is based on context information of the individual CpG islands within the nucleic acid sequence;
selecting one or more of the individual CpG islands for modification of one or more CpG pairs included in the one or more of the individual CpG islands;
for individual CpG islands as selected for modification of the one or more CpG pairs, identifying one or more alternative orders of nucleotides for the one or more CpG pairs, wherein the one or more alternative orders of nucleotides result in the synthesis of the same one or more compounds as the order of nucleotides;
analyzing the one or more alternative orders of nucleotides, wherein analyzing the one or more alternative orders of nucleotides includes determining one or more methylation probabilities for individual ones of the one or more alternative orders of nucleotides; and
presenting the one or more alternative orders of nucleotides.

13. The method of claim 12, wherein selecting the individual CpG islands is based on the one or more probabilities as determined.

14. The method of claim 12, wherein selecting the individual CpG islands is based on user-provided criteria.

15. The method of claim 12, further comprising:
for the individual CpG islands as detected, selecting a subset of CpG islands based on significance of downstream effects of methylation,
wherein determining the one or more probabilities of methylation is restricted to the subset of CpG islands.

16. The method of claim 12, wherein determining the one or more probabilities of methylation is further based on predictions generated by a model.

17. The method of claim 12, further comprising:
ranking the one or more alternative orders of nucleotides based on the analysis, and wherein presenting the one or more alternative orders of nucleotides is performed according to the ranking.

18. The method of claim 12, wherein identifying the one or more alternative orders of nucleotides for the one or more CpG pairs includes modifying and/or replacing one or more nucleotides in the one or more CpG pairs.

19. The method of claim 12, wherein the context information for an individual CpG island includes index information that indicates a start position and an end position within the nucleic acid sequence.

20. The method of claim 12, further comprising:
facilitating synthesis of some or all of the nucleic acid sequence, including at least one of the alternative orders of nucleotides as identified.
